(19) United States
(12) Patent Application Publication
(10) Pub. No.: US 2009/0232904 A1
(43) Pub. Date: Sep. 17, 2009

(54) Homeopathic Skin Care Compositions and Uses Thereof

(76) Inventors: Stephen Quinto, Pompano Beach, FL (US); Theo Quinto, Pompano Beach, FL (US); Benjamin Quinto, Pompano Beach, FL (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/403,855

(22) Filed: Mar. 13, 2009

Related U.S. Application Data
(60) Provisional application No. 61/036,381, filed on Mar. 13, 2008.

(51) Int. Cl. A61K 33/38; A61P 17/00; A61P 17/02; A61P 17/16

(52) U.S. Cl. 424/618

(57) ABSTRACT

The subject invention provides unique and advantageous compositions containing Argentum metallicum (homeopathic silver) for use in topical skin care. In one embodiment, the subject invention provides a gel formulation having an advantageous silver hydrosol component in addition to Argentum metallicum. In preferred embodiments of the composition of the subject invention, the formulation further comprises carbopol. Sodium hydroxide can be used as described herein to adjust the pH.

### CROSS-REFERENCE TO A RELATED APPLICATION

This application claims the benefit of U.S. provisional application Ser. No. 61/036,381, filed Mar. 13, 2008, which is incorporated herein by reference in its entirety.

### BACKGROUND OF INVENTION

The care and treatment of skin, including the treatment of wounds and irritations, is important for the prevention of infection, reduction of inflammation and pain, and the promotion of healing. Appropriate treatment also promotes the efficient preservation and/or restoration of cosmetic appearance and function.

The prevention of infection and control of inflammation is critical to effective skin care. For example, in the case of wounds, success in the prevention of wound infection directly affects the healing process and the degree to which one can obtain the other objectives.

The critical determinant or whether a wound will become infected is the number of bacteria present in a wound site, rather than bacterial type. There is experimental evidence that suggests a critical level of wound bacteria is approximately 10⁵ organisms per gram of tissue. Below this level, wounds can heal; at higher levels often result in infected wounds. Dirty wounds that have not been treated within six hours are likely to have bacterial contamination exceeding the critical level. Reducing the number of bacteria in and around the wound is a recognized and accepted means for avoiding infection and expediting wound healing.

There are a multitude of products available that can be applied to wounds that aid with one or all of these issues. However, many of these products or compositions include active ingredients that can have deleterious effects if applied incorrectly, have undesirable side effects, or can be dangerous if accidentally ingested, such as by children. Other remedies are effective at reducing infection, but have a stinging or burning sensation when applied to wounds.

Many people turn to homeopathic remedies and preparations in an effort to avoid the side effects and problems often associated with standard medicines. One of the basic tenets of homeopathic medicine is that a cure for a disease can be evoked by using a high dilution medicine. Homeopathy is widely accepted as a useful therapeutic throughout Europe, the British Commonwealth countries and India, and has been demonstrated to have characteristic and reproducible effects. A critical review of more than 100 controlled and/or clinical studies of homeopathy determined that patients received positive healing benefits from homeopathy beyond the placebo effect (Kleijnen, J. et al. 1991 Brit. Med. J. 302:316-323; Linde, K., Clausius, N., Ramirez, G., Melchart, D., Eitel, F., Hedges, L. V., Jonas, W. B., 1997, Lancet, 350:834-843; Reilly, D., et al, 1994, Lancet, 344:1601-1608).

For homeopathic medicines, after a base preparation is made, either by an extract or maceration of an herbal compound or the dissolving of a selected compound in a solvent, a series of dilutions are prepared from the initial batch, called the “mother tincture”. Homeopathic drugs are diluted according to either the decimal “X” or centesimal “C” scales. For a “3X” preparation, the mother tincture is diluted with nine parts of the desired diluent, in either liquid or powder form. The resultant mixture is then diluted a second time, in a ratio of one part mixture to ten parts solvent and the resulting mixture is diluted a third time in a ration of one to ten. Therefore, the 3X drug is actually at 10³ potency of the mother tincture. Similarly, a 6X dilution would be at 10⁶ potency of the original solution. In the “C scale” each dilution is done with ninety-nine parts diluent to the original mixture. Therefore, a 3 C solution is at 10⁶ potency of the original mixture and thus corresponds to a 6X potency. These scales are recognized by the Homeopathic Pharmacopeia of the United States (H.P.U.S.).

Many homeopathic medicines are used at concentrations of micrograms (10⁶ M) and nanograms (10¹² M); however, in other homeopathic preparations, the dilutions exceed Avogadro's number (6.023×10²³). When compounds are diluted 1:10 (or 1:100), with repeated succusions (violent shaking or pounding) and repetitively diluted by this procedure these compounds are prepared and labeled as homeopathic remedies.

Silver and related compounds have long been recognized as anti-microbial agents and are often used as such in homeopathic remedies. Argentum metallicum is form of homeopathic silver. However, the effectiveness of silver is greatly dependent upon its formulation.

### BRIEF SUMMARY

The subject invention provides unique and advantageous compositions containing Argentum metallicum (homeopathic silver) for use in topical skin care. In one embodiment, the subject invention provides a gel formulation having an advantageous silver hydrosol component in addition to Argentum metallicum. In preferred embodiments of the composition of the subject invention, the formulation further comprises carbopol. Sodium hydroxide can be used as described herein to adjust the pH.

In one embodiment, the silver hydrosol component can be a silver colloidal composition containing silver in a picoscalar particle size. Advantageously, the compositions of the subject invention provide functional silver ions in safe, effective and non-toxic formulations.

The invention further provides methods of use for the new homeopathic compositions. Thus, in one embodiment, the gel can be used for the care and treatment of skin wounds and irritations. In a related embodiment, the gel is an antimicrobial that can inhibit the proliferation of bacteria in and around skin wounds and irritations.

In specific embodiments, the compositions of the subject invention can be used for relief of, for example, topical burning, stinging, itching, redness, stiffness and pain from minor wounds, burns, bruises, ulcerations, sunburn, razor burn, scrapes, rashes, blisters, bug bites, warts, cellulitus, and skin eruptions from acne, eczema or infection.

Advantageously, the compositions can be used to reduce pain and inflammation, and are safe for use on sensitive skin.

The subject invention also provides methods for formulating Argentum metallicum and a silver hydrosol into gel form.

### DETAILED DISCLOSURE

The subject invention provides unique compositions containing Argentum metallicum (homeopathic silver) for use in topical skin care. In one embodiment, the subject invention provides a gel formulation having an advantageous silver hydrosol component in addition to Argentum metallicum. In preferred embodiments, the formulation further comprises carbopol. Sodium hydroxide can be used as described herein to adjust the pH.

In one embodiment, the silver hydrosol component can be a silver colloidal system containing silver in a picoscalar particle size. The dispersion medium is water. As understood by those skilled in the art, a colloidal system is an intimate mixture of two substances, one of which, called the dispersed phase (or colloid), is uniformly distributed in a finely divided state through the second substance, called the dispersion medium (or dispersing medium). The compositions of the subject invention provide functional silver hydrosol ions and Argentum metallicum in safe, effective and non-toxic formulations.

In accordance with the subject invention, the introduction of impurities or extraneous substances is carefully avoided both during the manufacturing process and storage to avoid a separation of the dispersed phase of silver from the dispersing medium, usually water.

In one embodiment, the gel can be used for the care and treatment of skin wounds and irritations. In a further embodiment, the gel is an antimicrobial that can inhibit the proliferation of bacteria in and around skin wounds and irritations.

The compositions of the subject invention can be used for relief of, for example, topical burning, stinging, itching, redness, stiffness and pain from minor wounds, burns, bruises, ulcerations, sunburn, razor burn, scrapes, rashes, blisters, bug bites, and skin eruptions from acne, eczema or infection.

In one embodiment, the compositions of the subject invention helps the body fight skin infections, thereby facilitating and expediting the healing process for minor wounds. These wounds include, for example, scrapes and abrasions.

In a further embodiment, the composition of the subject invention calms inflammation and reduces the swelling and redness that can accompany a wound.

In yet a further embodiment, the compositions of the subject invention can be used to reduce the pain that can accompany skin wounds and irritations.

The homeopathic preparations of the present invention are non-toxic and do not produce undesirable side effects. They can be formulated and provided to a large patient population at a reasonable cost by means of delivery systems that are convenient and safe. Homeopathic preparations of the present invention are preferably administered via topical delivery systems.

Advantageously, the compositions of the subject invention have an excellent shelf life and do not require preservatives.

The subject invention further provides a method for formulating Argentums metallicum and a silver hydrosol into gel form.

### DEFINITIONS

As used herein the term “effective amount” refers to the amount of the subject composition required to confer a prophylactic or therapeutic effect on a treated subject.

The terms “patient” or “subject” as used herein, describes an animal, including mammals to which the materials and methods of the present invention are applied. Mammalian species that can benefit from the disclosed materials and methods include, but are not limited to, humans, domesticated animals (e.g. pets) such as dogs, cats, guinea pigs, hamsters; and veterinary uses for large animals such as cattle, horses, goats, sheep. Preferably, the animal is a human.

### Compositions

In one embodiment, the formulation of the subject invention comprises Argentum metallicum and a silver hydrosol formulated with a crosslinked acrylic acid-based polymer, such as, for example, Carbopol®.

In specific embodiments, the compositions of the subject invention can comprise a combination of Argentums metallicum including, for example, Argentums metallicum 10×HPUS, Argentums metallicum 20×HPUS, and Argentums metallicum 30×HPUS. As would be appreciated by those skilled in the art, “HPUS” indicates that this ingredient is officially a part of the Homeopathic Pharmacopoeia of the United States, which is incorporated herein by reference.

The concentration of Argentum metallicum can be, for example, between 0.5% and 15%, preferably between 1% and 10% and most preferably between 2% and 5%. The concentration of the silver hydrosol can be, for example, from 50% to 99%, preferably from 80% to 98.5% and most preferably from 95% to 98%. The concentration of carbopol (which is preferably food grade) can be, for example, from 0.1% to 5% and is preferably from 0.2% to 1%.

Gels are the preferred vehicles of dispensing the homeopathic formulations of the invention. Gels maintain a uniformity that is useful in keeping active ingredients in the homeopathic formulations evenly dispersed in their aqueous base. Brownian motion builds up networks in gels and restores their shape when they have been ruptured by stresses (e.g. settling or shaking). The longer the dosage contacts the skin (i.e. nerve endings), the longer the duration of action. As the preferred formulation of the invention is in a gel base that soothes and smoothes the skin without socially objectionable odor, one is less likely to want to wash away the homeopathic formulation from the skin.

In addition to the preferred gel base, various other gel base formulations can carry the active ingredients in the homeopathic formulations of the invention with varying degrees of success. However, the use of glycerine and calendula and other homeopathics in a water gel base of the preferred formulation is very emollient, non-drying, and soothing to the skin. Other carrier bases may also be used to deliver the active ingredients topically such as: water, alcohol, water/alcohol, cream ointment, salves, lotion, liniment, tinctures, cream gel, lotion ointment, rub, spray, aerosol, lotion spray, balm rub, gel ointment, lotion cream, poultice, plaster, infusion, decoction and other herbal methods of preparation. However, the gel delivery system is preferred.

Advantageously, in preferred embodiments, the compositions of the subject invention are non-greasy, transparent and odorless. A further advantage of the compositions of the subject invention is that they do not have side-effects and are contemplated for sale over-the-counter (OTC).

### Methods and Methods

The subject invention further provides a method for producing a formulation containing Argentum metallicum and a silver hydrosol gel. Preferably, carbopol is also included in the formulation and sodium hydroxide is used to adjust the pH.

Silver hydrosol is a formulation known in the art as ultra-pure/ultra-small silver ion particles dispersed in ultra-pure water. Silver hydrosol can be produced by an electrolytic process that liberates silver particles of, for example, picoscalar size. The ionic charge on the silver in silver hydrosol is uniformly positive, which aids in its uniform dispersion in a pure water medium. Because the potential for biological activity is directly affected by the formulation, the ultra-fine dispersion of silver in silver hydrosol can result in enhanced bioavailability.

Silver hydrosol, such as, for example, Argentyn 23, is known in the art as a formulation of ultra-pure/ultra-small silver ion particles dispersed in ultra-pure water. Silver hydrosol can be produced by an electrolytic process that liberates silver particles of picoscalar size, equivalent to approximately 0.8 nanometers (0.0008 microns). The ionic charge on the silver in silver hydrosol is uniformly positive, which aids in its uniform dispersion in a pure water medium. The potential for biological activity is directly affected by the formulation. Thus, the ultra-fine dispersion of silver in silver hydrosol can result in enhanced bioavailability.

The introduction of impurities or extraneous substances can result in a separation or agglomeration of the dispersed phase of silver from the dispersing medium, i.e. water. Other non-material factors can cause agglomeration of silver hydrosol products. Thus, all of these factors should be avoided in the preparation of the formulation of the subject invention.

### Applications

In one embodiment the gel can be use as a treatment for wounds and irritations of the skin. In a further embodiment, the gel can be used as an antimicrobial agent to inhibit proliferation of microbes in and around skin wounds and irritations, such as, for example, burns, stings, itch, swelling, ulcerations (cuts and scrapes), blisters, bug bites, skin eruptions from acne, eczema, or infections, and rashes. The active compound can be applied directly to an affected area on any part of the skin.

Many currently known OTC products for topical uses have significant drawbacks. There is a need for an OTC topical product without unpleasant physical and social side effects such as strong odors, counterirritation, redness, itching, stinging, cooling, sensitization, staining, burning, perfumes, anesthesia, etc. Ideally such product would also quickly relieve inflammation, edema, redness, and swelling along with pain.

An examination of existing OTC topical pain products in most pharmacies reveals, that although there are many brands to choose from, they all basically use various combinations of the same active ingredients, namely: menthol, methyl salicylate, camphor, and trolamine salicylate which have the drawbacks mentioned above. There is a need for novel formulations having anti-microbial, analgesic and/or anti-inflammatory properties that can avoid the drawbacks of the prior art products. The homeopathic formulations of the present invention fulfill such a need. The formulations of the invention are not counterirritants and do not rely upon massage, heat, stimulation or counterirritation to allay pain. The formulations also do not require a trained homeopath. Furthermore, the compositions do not sting, and are safe for use with children and others with sensitive skin.

The following examples illustrate procedures for practicing the subject invention. This example is provided for the purpose of illustration only and should not be construed as limiting. Thus, any and all variations that become evident as a result of the teachings herein or from the following examples are contemplated to be within the scope of the present invention.

### Example 1

The compositions of the subject invention can be used by, for example, cleaning the effected area of the skin followed by liberal application of the gel of the subject invention. This can be repeated, for example, three times per day or as after as is needed to relieve symptoms. In preferred embodiments, the effected area is left wet and/or bandaged.

### Example 2

### Method for Manufacturing

Preparation of this gel should be done with the utmost care. Contaminants introduced into the product can cause mass agglomeration, thus destroying the effectiveness of the product.
Other non-material factors that can cause agglomeration and destruction of the product as well.
   The materials utilized for this procedure include:

### Argentyn Metallicum

### Argentyn (Natural Immunogenics Corp.)

Food grade Carbopol

### 1 N Sodium Hydroxide

The method disclosed herein results in a composition having a final composition of the following ratio:

0.50% Carbopol 974P

3.0% Argentyn Metallicum

96% Argentyn silver hydrosol,

approximately 0.05%-0.10% 1N Sodium hydroxide)

Upon combining these ingredients, a composition of the subject invention is obtained.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

It should also be understood that any reference in this specification to “one embodiment,” “an embodiment,” “example embodiment,” “further embodiment,” “alternative embodiment,” etc., is for literary convenience. The implication is that any particular feature, structure, or characteristic described in connection with such an embodiment is included in at least one embodiment of the invention. The appearance of such phrases in various places in the specification does not necessarily refer to the same embodiment. Further, when a particular feature, structure, or characteristic is described in connection with any embodiment, it is submitted that it is within the purview of one skilled in the art to affect such feature, structure, or characteristic in connection with other ones of the embodiments.

The invention has been described herein in considerable detail, in order to comply with the Patent Statutes and to provide those skilled in the art with information needed to apply the novel principles, and to construct and use such specialized components as are required. However, it is to be understood that the invention can be carried out by specifically different materials and procedures, and that various modification, both as to equipment details and operating procedures can be effected without departing from the scope of the invention itself. Further, it should be understood that, although the present invention has been described with reference to specific details of certain embodiments thereof, it is not intended that such details should be regarded as limitations upon the scope of the invention except as and to the extent that they are included in the accompanying claims.

We claim:

1. A homeopathic skin care gel composition for topical administration comprising Argentum metallicum and silver hydrosol.

2. The composition, according to claim 1, further comprising carbopol.

3. The composition, according to claim 2, consisting essentially of Argentum metallicum, silver hydrosol, and carbopol.

4. A method for improving the health of skin wherein said method comprises administering to the skin of a patient an effective amount of a composition of claim 1.

5. The method, according to claim 4, used to treat pain.

6. The method, according to claim 4, used to treat inflammation.

7. The method, according to claim 4, used to treat topical burning, stinging, itching, redness, stiffness and pain from minor wounds, burns, bruises, ulcerations, sunburn, razor burn, scrapes, rashes, blisters, bug bites, or skin eruptions from acne, eczema or infection.
